(19) United States
(12) Patent Application Publication
(10) Pub. No.: US 2019/0183335 A1
(43) Pub. Date: Jun. 20, 2019

(54) OPHTHALMIC SURGICAL MICROSCOPE IMAGE INVERTER

(71) Applicant: Novartis AG, Basel (CH)

(72) Inventors: Lingfeng Yu, Rancho Santa Magarita, CA (US); Valentina Doushkina, Aliso Viejo, CA (US)

(21) Appl. No.: 16/216,497

(22) Filed: Dec. 11, 2018

Related U.S. Application Data
(60) Provisional application No. 62/607,721, filed on Dec. 19, 2017.

(51) Int. Cl. A61B 3/13; G02B 21/00; G02B 5/04

(52) U.S. Cl. CPC A61B 3/13; G02B 5/04; G02B 21/0012

(57) ABSTRACT

The present disclosure relates to an ophthalmic surgical microscope including a magnifying lens positioned in an optical path, a reflection inverter positioned in the optical path, and an ocular lens or eyepiece positioned in the optical path. The reflection inverter may include A Schmidt-Pechan prism or a pair of inverting lenses and a reduction lens.

### TECHNICAL FIELD

The present disclosure relates to ophthalmic surgery, and more specifically, to an ophthalmic surgical microscope containing a reflection inverter for inverting an image of an eye, particularly an eye undergoing ophthalmic surgery.

### BACKGROUND

Ophthalmic surgery saves and improves the vision of tens of thousands of patients every year. However, given the sensitivity of vision to even small changes in the eye and the minute and delicate nature of many eye structures, ophthalmic surgery is difficult to perform and the reduction of even minor or uncommon surgical errors or modest improvements in accuracy of surgical techniques can make an enormous difference in the patient's vision after the surgery.

One type of ophthalmic surgery, vitreoretinal surgery, encompasses various delicate procedures involving internal portions of the eye, such as the vitreous humor and the retina. Different vitreoretinal surgical procedures are used, sometimes with lasers, to improve visual sensory performance in the treatment of many eye diseases, including epimacular membranes, diabetic retinopathy, vitreous hemorrhage, macular hole, detached retina, and complications of cataract surgery, among others.

During ophthalmic surgery, such as vitreoretinal surgery, an ophthalmologist typically uses a surgical microscope to view a magnified image of the eye undergoing surgery. Depending on the surgical microscope and optical system used, the magnified image viewed by the surgeon may be inverted as compared to the actual eye. For example, the eye, as it appears in the magnified image may be upside down or with left and right reversed as compared to the actual eye. The surgeon must then mentally correct the image in order to move surgical instruments in the eye properly, which can negatively impact the surgical outcome.

### SUMMARY

The present disclosure provides an ophthalmic surgical microscope including a magnifying lens positioned in an optical path, a reflection inverter positioned in the optical path, and an ocular lens or eyepiece positioned in the optical path.

The ophthalmic surgical microscope may further be combined with any of the following features or any other features described in this specification, shown in the figures, or both, all of which may also be combined with one another unless clearly mutually exclusive:

i) the ophthalmic surgical microscope may have a stack height equal to or less than 5% longer than an otherwise identical ophthalmic surgical microscope lacking the reflection inverter;
ii) the reflection inverter may include a Schmidt-Pechan prism in the optical path, and the Schmidt-Pechan prism may be operable to invert an image traveling along the optical path;
ii-a) the Schmidt-Pechan prism may reduce the length of the optical path outside the Schmidt-Pechan prism by at least 20%;
ii-b) the Schmidt-Pechan prism may be operable to reflect light in the visible wavelengths internally at internal angles of incidence that are each 90° or less;
ii-c) the Schmidt-Pechan prism may be operable to exhibit at least 98% internal reflection of incident light in the visible wavelengths;
ii-d) the Schmidt-Pechan prism may exhibit internal reflection of at least 80% of a field of view of the ophthalmic surgical microscope;
ii-e) the Schmidt-Pechan prism may be made from a material transparent to the visible wavelengths of light and having an Abbe number of at least 40;
ii-f) the Schmidt-Pechan prism may be made from a material transparent to the visible wavelengths of light and having a reflective index of at least 1.40;
ii-g) the reflection inverter may be operable to preserve the polarization of any light in the visible wavelengths traveling along the optical path;
ii-h) the ophthalmic surgical microscope may not include a reduction lens in the optical path.
iii) the reflection inverter may include a right angle reflector having a first reflective face and a second reflective face, both in the optical path, a first inverting lens in the optical path, and a second inverting lens in the optical path, wherein the first reflective face of the right angle reflector may be operable to reflect light traveling along the optical path such that the light strikes and is transmitted by the first inverting lens then strikes the first reflector at a first angle of incidence, wherein the first reflector may be operable to reflect light at an angle 90° with respect to the first angle of incidence along the optical path such that the light then strikes the second reflector at a second angle of incidence, wherein the second reflector may be operable to reflect light at an angle 90° with respect to the second angle of incidence along the optical path such that the light then strikes and is transmitted by the second inverting lens, and then strikes the second reflective face of the right angle reflector, wherein the second reflective face of the right angle reflector may be operable to reflect the light along the optical path in a direction that is collinear with a direction in which the light struck the first reflective face, and wherein the reflection inverter may be operable to invert an image traveling along the optical path;
iii-a) the right angle reflector may include a right angle prism mirror;
iii-b) the first reflective face and the second reflective face of the right angle reflector may both be operable to reflect at least 90% of incident light in the visible wavelengths;
iii-c) the first inverting lens and the second inverting lens may both be made from a material transparent to the visible wavelengths of light and having an Abbe number of at least 40;
iii-d) the first inverting lens and the second inverting lens may both be made from a material transparent to the visible wavelengths of light and having a reflective index of at least 1.40;
iii-e) the microscope may further include a reduction lens in the optical path that may be operable to reduce the optical path by at least 20%;
iii-f) the reduction lens may be made from a material transparent to the visible wavelengths of light and having an Abbe number of at least 40;
iii-g) the reduction lens may be made from a material transparent to the visible wavelengths of light and having a reflective index of at least 1.40; and
iii-h) the reduction lens may include a biconcave lens, a biconvex lens, a convex-concave lens, a plano concave lens, a plano convex lens, a positive/negative meniscus lens, an aspheric lens, a converging lens, a diverging lens, a liquid crystal lens, a diffractive lens, and any combinations thereof.

### BRIEF DESCRIPTION OF THE DRAWINGS

For a more complete understanding of the present invention and its features and advantages, reference is now made to the following description, taken in conjunction with the accompanying drawings, in which:

FIG. 1 is schematic diagram of an ophthalmic surgical microscope containing an inverter;

FIG. 2 is a set of schematic diagrams of a two views of Schmidt-Pechan prism reflection inverter suitable for use in the ophthalmic surgical microscope of FIG. 1; internal prism angles are indicated in the left diagram;

FIG. 3 is a schematic diagram of a mirror/inverting lens reflection inverter suitable for use in the ophthalmic surgical microscope of FIG. 1; and

FIG. 4 is a schematic diagram of how an image is inverted using the mirror/inverting lens reflection inverter or FIG. 3.

### DETAILED DESCRIPTION

The present disclosure relates to ophthalmic surgery, and more specifically, to an ophthalmic surgical microscope containing a reflection inverter. Such an ophthalmic surgical microscope, by providing a magnified image of the eye undergoing surgery that is not inverted as compared to the eye, may decrease the difficulty of ophthalmic surgery an improve surgical outcomes for patients. In addition, the ophthalmic surgical microscope may have a stack height comparable to conventional ophthalmic surgical microscopes and, may, therefore, preserve ergonomic usability of the surgical microscope. In particular, the ophthalmic surgical microscope with the reflection inverter may have a stack height equal to or less than 5% longer, less than 2% longer, or less than 1% longer than an otherwise identical ophthalmic surgical microscope lacking the reflection inverter.

It will be understood that the component described herein as having certain properties with respect to light are operable to exhibit those properties when in the presence of light, such as during use of the ophthalmic surgical microscope.

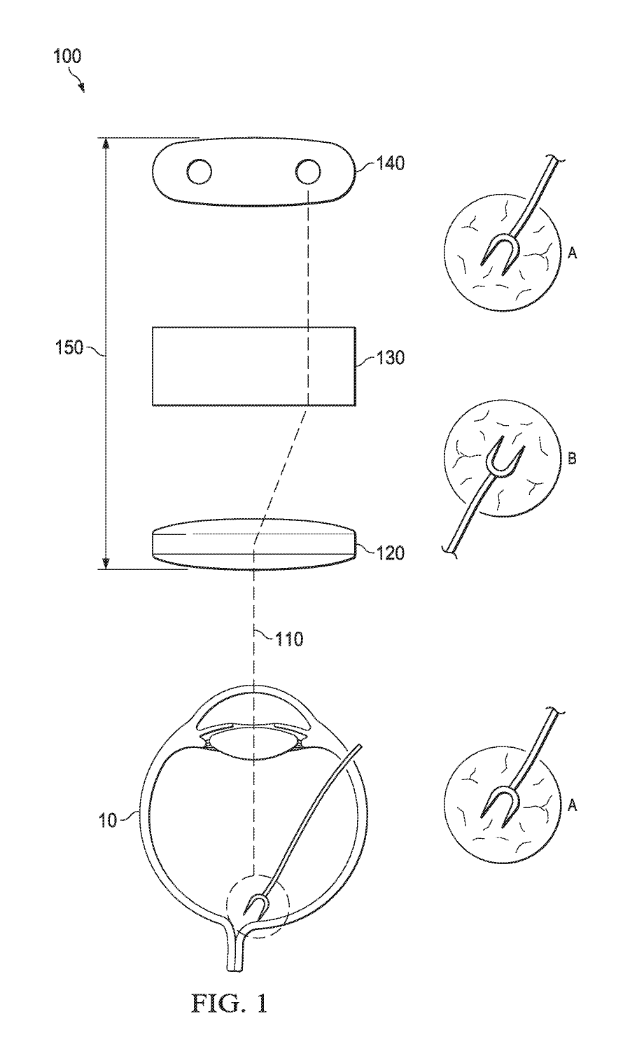

FIG. 1 is a schematic diagram of an ophthalmic surgical microscope 100. The ophthalmic surgical microscope 100 includes at least one magnifying lens 120 that is positioned in an optical path 110 (dotted line) that also extends through a reflection inverter 130 to an ocular lens or eyepiece 140, which may be selectively positionable so that a portion of the eye 10 viewed by using the ophthalmic surgical microscope is in focus. Portions of the eye suitable for viewing can include the retina, macula, foveola, fovea centraalis, para fovea, perifovea, optic disc, optic cup, one of more layers of the retina, vitreous, vitreous body, or any portion in which a surgical instrument is also viewed.

During use of the surgical microscope 100 to view an eye 10, the optical path 110 further extends to the eye 10 being viewed. The ophthalmic surgical microscope 100 also has a stack height, 150, which is generally parallel to the optical path 110, and which may extend from the portion of the ophthalmic surgical microscope 100 in the optical path 110 that is closest to the eye 10 during use through the ocular lens or eyepiece 140. The stack height 150, may be comparable to that of a similar ophthalmic surgical microscope containing only a reduction lens and lacking a reflection inverter 130.

Due to the inverter 130 being a reflection inverter, light transmitted by the reflection inverter 130 has an axis that is collinear with and in the same direction as light incident on the reflection inverter 130.

A portion of the eye 10, which is shown upright with a surgical tool also present in position “A,” may be inverted when it passes through the magnifying lens 120, to position “B.” Although ophthalmic surgeons can successfully operate even when viewing an inverted image in position “B,” doing so requires further mental processing and may increase surgery time or increase the chance of a less positive or even a negative surgical outcome. Accordingly, a reflection inverter 130 may be included in the ophthalmic surgical microscope 100 to invert the image back to position “A” as shown in FIG. 1. In addition, although various instruments exist that may invert an image, the reflection inverter 130 in ophthalmic surgical microscope 100 does so without an unacceptable increase in stack height or loss of image quality.

The ophthalmic surgical microscope 100 may further include other general components, such as additional lenses, mirrors, such as dichroic mirrors, digital light capture equipment, such as a digital camera, digital displays, and a processor programmed to process data from digital light capture equipment and display it on a digital display, or to insert images in a display, including in a display seen through ocular lens or eyepiece 140. The ocular lens or eyepiece 140 may include a zoom lens, a liquid crystal lens, or any other suitable variable focal length lens, or a fixed focus length lens.

The ophthalmic surgical microscope 100 may also include other components specific for a particular procedure, such as a contact lens, particularly a macular contact lens, that may be coupled to the eye 10, or an optical coherence tomography (OCT) system. A processor, if present, may further be programmed to perform functions associated with these more specific components, such as analysis of OCT data to create a two-dimensional or three-dimensional OCT image and to display the image on a digital display or to insert the image into a display.

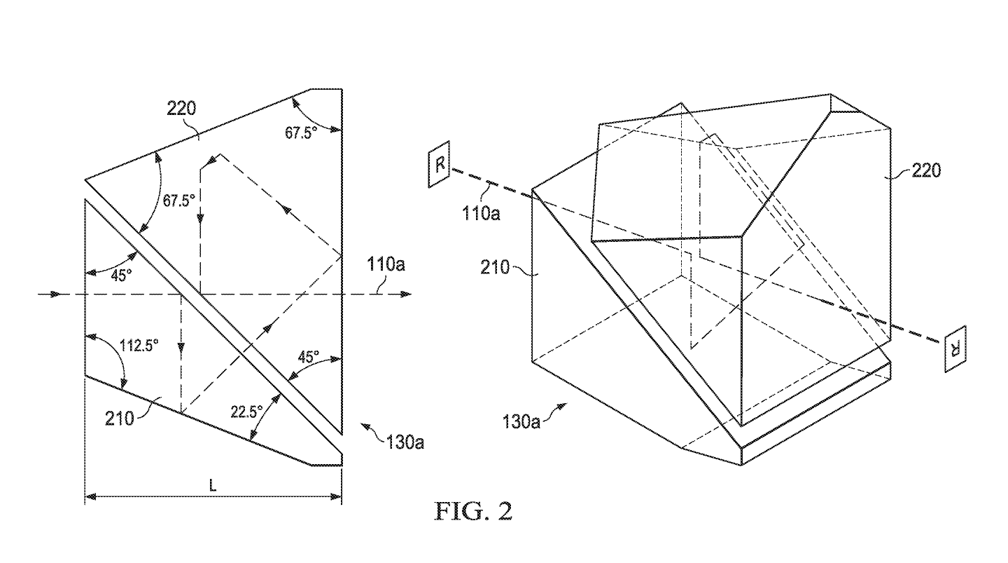

The reflection inverter 130, in one embodiment, may include a Schmidt-Pechan prism reflection inverter 130a as illustrated in FIG. 2. A light beam entering the Schmidt-Pechan prism reflection inverter 130a travels along optical path 110a. In the process, any image contained in the light beam is inverted, as indicated by the letter “R” in the diagram.

The Schmidt-Pechan prism reflection inverter 130a may be made from two prisms, 210 and 220, positioned with respect to one another generally as indicated in FIG. 2. The Schmidt-Pechan prism configuration, unlike many other inverting prisms configurations, is mechanically stable and will not tend to cause the microscope to wobble or tip in any direction once mounted in the ophthalmic surgical microscope 100. Furthermore, the optical path 110 in ophthalmic surgical microscope 100 often extends too long before an image of the eye 10 is in focus. A reduction lens is often currently used to shorten the total optical path 110 from the eye 10 to the ocular lens or eyepiece 140 so that the stack height 150 of the ophthalmic surgical microscope 100 is not too long. A reflection inverter 130a containing a Schmidt-Pechan prism has a relatively long internal optical path, 110a, within a short total length, L of the prism, such that a long optical path 110 can be accommodated by an ophthalmic surgical microscope 100 without a stack height 150 that is too high. The Schmidt-Pechan prism may effectively reduce the length of the optical path 110 outside of the Schmidt-Pechan prism by at least 20%, at least 30%, at least 40%, or at least 50%.

The Schmidt-Pechan prism in the reflection inverter 130a reflects light internally at internal angles of incidence as shown by the optical path 110a in FIG. 2. Because each internal angle of incidence is 90° or less, it may be possible to achieve at least 98% internal reflection of incident light in the visible wavelengths (390 to 700 nm), at least 99% internal reflection of incident light in the visible wavelengths, at least 99.5% internal reflection of incident light in the visible wavelengths, at least 99.9% internal reflection of incident light in the visible wavelengths, or even 100% internal reflection of incident light in the visible wavelengths (referred to as total internal reflection). This internal reflection may be for the entire field of view of the ophthalmic surgical microscope 100, or for at least 80%, at least 90%, at least 95%, or at least 99% of the field of view.

The Schmidt-Pechan prism in the reflection inverter 130a may also be made from a material transparent to the visible wavelengths, such as a glass, having a high Abbe number in the visible wavelengths. The Abbe number, also referred to as the V-number, is a measure of the material's dispersion, or the variation of refractive index versus wavelength. In particular, the material may have an Abbe number of at least 40, at least 45, at least 50, or at least 60. If the material is a flint glass, it may have an Abbe number between 50 and 55. If the material is a crown glass, it may have an Abbe number of between 50 and 85.

The Schmidt-Pechan prism in the reflection inverter 130a may also be made from a material transparent to the visible wavelengths, such as a glass, also having a high reflective index in the visible wavelengths. The reflective index describes how fast light propagates through a material as compared to in a vacuum and is found using the equation n=c/v, where n is the reflective index, c is the speed of light, and v is the phase velocity of light in the material. In particular, the material may have a reflective index of at least 1.40, at least 1.45, or at least 1.50. If the material is a flint glass, it may have a reflective index of at least 1.50, such as approximately 1.52. If the material is a crown glass, it may have a reflective index of between 1.45 and 2.00.

Further, the Schmidt-Pechan prism in the mirror/inverting lens reflection inverter 130a may preserve any polarization of light in the visible wavelengths that passes through the reflection inverter 130a along the optical path 110a.

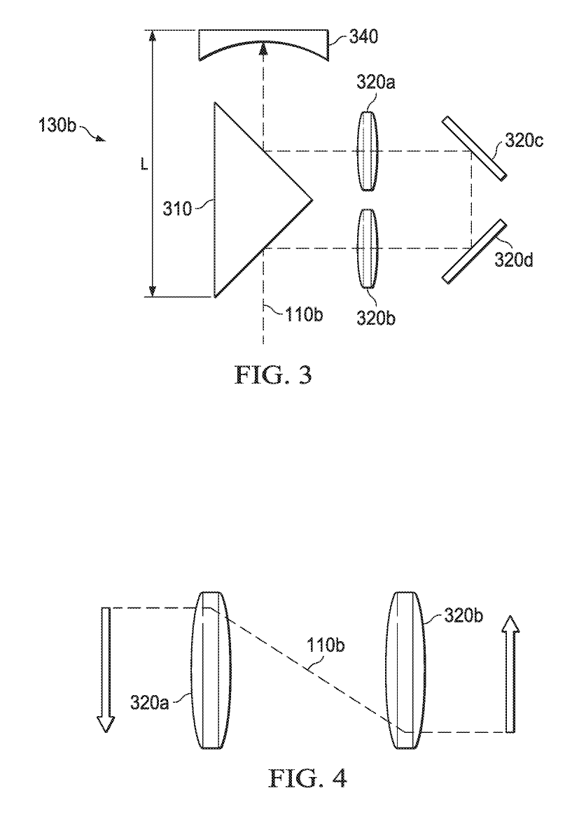

The reflection inverter 130, in one embodiment, may be a reflection inverter 130b shown in FIG. 3. The reflection inverter 130b includes a right angle reflector, such as the right angle prism mirror 310 illustrated. Other objects containing two reflective faces at right angles to one another and position with respect to the other components of the reflection inverter 130b as shown in FIG. 3 may also be used in place of the right angle prism mirror 310. Light traveling along the optical path 110b strikes a first face of the right angle prism mirror 310 and is reflected to a first inverting lens 320a, then strikes a first reflector 320c. The light strikes the first reflector 320c at a first angle of incidence and is reflected from the first reflector 320c at angle that is 90° with respect to the first angle of incidence. The light reflected from the first reflector 320c then strikes the second reflector 320d. The light strikes the second reflector 320d at a second angle of incidence and is reflected from the second reflector 320d at angle that is 90° with respect to the second angle of incidence. The second reflector 320d also reflects the light at an angle that is 90° with respect to the second angle of incidence. The light then strikes the second inverting lens 320b. The light transmitted by the second inverting lens 320b then strikes a second reflective face of the right angle prism mirror 310 and is reflected along an optical path in a direction that is collinear with the direction in which light struck the first reflective face of the right angle prism mirror 310.

While traveling through the inverting lenses 320a and 320b, an image contained in the light is inverted as illustrated in FIG. 4. Two (as shown in FIG. 3.) or more reflectors can be added to redirect the beam as desired and form an inverted image.

Because the portion of the optical path 110a through the right angle prism mirror 310 and the inverting lenses 320a and 320b is typically short it does not significantly reduce the total optical path 110 within the ophthalmic surgical microscope 100. Accordingly, a reduction lens 340 may further be included, which may be fixed or selectively positionable to change the focus position such that the a portion of the eye 10 may be viewed in focus. The reduction lens 340 can include one or more optical components, such as a biconcave lens, a biconvex lens, a convex-concave lens, a plano concave lens, a plano convex lens, a positive/negative meniscus lens, an aspheric lens, a converging lens, a diverging lens, a liquid crystal lens, a diffractive lens, other suitable lenses, and any combinations thereof.

The reduction lens 340 may reduce the length of the optical path 110 by at least 20%, at least 30%, at least 40%, or at least 50%.

The total length, L between the edge of the reduction lens 340 closes to the ocular lens or eyepiece 140 and the edge of the right angle prism mirror or first inverting lens 320a, whichever is further, that is furthest from the reduction lens 340, may be such that the stack height of the ophthalmic surgical microscope 100 is not too long.

The right angle prism mirror or other right angle reflector may reflect, on both faces, at least 90%, at least 95%, at least 99%, or at least 99.9% of incident light in the visible wavelengths. Similarly, both of inverting lenses 320 may transmit at least 90%, at least 95%, at least 99%, or at least 99.9% of incident light in the visible wavelengths. In addition, both of inverting lenses may have Abbe numbers and reflective indices the same as those described above for the Schmidt-Pechan prism and may be made of the same materials. Similarly, the reduction lens 340 may transmit at least 90%, at least 95%, at least 99%, or at least 99.9% if incident light in the visible wavelengths and may have Abbe numbers and reflective indices the same as those described above for the Schmidt-Pecham prism and may be made of the same materials. Polarization may also be preserved.

An ophthalmic surgical microscope 100 as described herein may be used in any sort of ophthalmic surgery in which a portion of an eye 10 is magnified. Typically magnification may be between 5x and 40x, more particularly between 5x and 15x. The ophthalmic surgical microscope 100 may not only provide an image of the eye 10 through the ocular lens or eyepiece 140, it may be coupled with other microscope components or a processor to provide the same image on a digital viewed, such as a screen used by an assistant, to provide an digitially enhanced image of the eye 10, to provide additional information, such as OCT image, or any combinations of these images and any other images presented to the surgeon performing ophthalmic surgery, any assistants, or both.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. An ophthalmic surgical microscope comprising:
a magnifying lens positioned in an optical path;
a reflection inverter positioned in the optical path; and
an ocular lens or eyepiece positioned in the optical path.

2. The ophthalmic surgical microscope of claim 1, wherein the ophthalmic surgical microscope has a stack height equal to or less than 5% longer than an otherwise identical ophthalmic surgical microscope lacking the reflection inverter.

3. The ophthalmic surgical microscope of claim 1, wherein the reflection inverter comprises a Schmidt-Pechan prism in the optical path, wherein the Schmidt-Pechan prism is operable to invert an image traveling along the optical path.

4. The ophthalmic surgical microscope of claim 3, wherein the Schmidt-Pechan prism may reduce the length of the optical path outside the Schmidt-Pechan prism by at least 20%.

5. The ophthalmic surgical microscope of claim 3, wherein the Schmidt-Pechan prism is operable to reflect light in the visible wavelengths internally at internal angles of incidence that are each 90° or less.

6. The ophthalmic surgical microscope of claim 3, wherein the Schmidt-Pechan prism is operable to exhibit at least 98% internal reflection of incident light in the visible wavelengths.

7. The ophthalmic surgical microscope of claim 3, wherein the Schmidt-Pechan prism exhibits internal reflection of at least 80% of a field of view of the ophthalmic surgical microscope.

8. The ophthalmic surgical microscope of claim 3, wherein the Schmidt-Pechan prism is made from a material transparent to the visible wavelengths of light and having an Abbe number of at least 40.

9. The ophthalmic surgical microscope of claim 3, wherein the Schmidt-Pechan prism is made from a material transparent to the visible wavelengths of light and having a reflective index of at least 1.40.

10. The ophthalmic surgical microscope of claim 3, wherein the reflection inverter is operable to preserve the polarization of any light in the visible wavelengths traveling along the optical path.

11. The ophthalmic surgical microscope of claim 3, wherein the ophthalmic surgical microscope does not comprise a reduction lens in the optical path.

12. The ophthalmic surgical microscope of claim 1, wherein the reflection inverter comprises:
a right angle reflector having a first reflective face and a second reflective face, both in the optical path;
a first inverting lens in the optical path;
a second inverting lens in the optical path, a first reflector in the optical path; and
a second reflector in the optical path,
wherein the first reflective face of the right angle reflector is operable to reflect light traveling along the optical path such that the light strikes and is transmitted by the first inverting lens, then strikes the first reflector and is reflected at a first angle of incidence,
wherein the first reflector is operable to reflect light at an angle 90° with respect to the first angle of incidence along the optical path such that the light then strikes the second reflector at a second angle of incidence,
wherein the second reflector is operable to reflect light at an angle 90° with respect to the second angle of incidence along the optical path such that the light then strikes and is transmitted by the second inverting lens, then strikes the second reflective face of the right angle reflector,
wherein the second reflective face of the right angle reflector is operable to reflect the light along the optical path in a direction that is collinear with a direction in which the light struck the first reflective face, and
wherein the reflection inverter is operable to invert an image traveling along the optical path.

13. The ophthalmic surgical microscope of claim 12, wherein the right angle reflector comprises a right angle prism mirror.

14. The ophthalmic surgical microscope of claim 12, wherein the first reflective face and the second reflective face of the right angle reflector are both operable to reflect at least 90% of incident light in the visible wavelengths.

15. The ophthalmic surgical microscope of claim 12, wherein the first inverting lens and the second inverting lens are both made from a material transparent to the visible wavelengths of light and having an Abbe number of at least 40.

16. The ophthalmic surgical microscope of claim 12, wherein the first inverting lens and the second inverting lens are both made from a material transparent to the visible wavelengths of light and having a reflective index of at least 1.40.

17. The ophthalmic surgical microscope of claim 12, further comprising a reduction lens in the optical path operable to reduce the optical path by at least 20%.

18. The ophthalmic surgical microscope of claim 17, wherein the reduction lens is made from a material transparent to the visible wavelengths of light and having an Abbe number of at least 40.

19. The ophthalmic surgical microscope of claim 17, wherein the reduction lens is made from a material transparent to the visible wavelengths of light and having a reflective index of at least 1.40.

20. The ophthalmic surgical microscope of claim 17, wherein the reduction lens comprises a biconcave lens, a biconvex lens, a convex-concave lens, a plano concave lens, a plano convex lens, a positive/negative meniscus lens, an aspheric lens, a converging lens, a diverging lens, a liquid crystal lens, a diffractive lens, and any combinations thereof.
